(19) United States
(12) Patent Application Publication
(10) Pub. No.: US 2009/0069818 A1
(43) Pub. Date: Mar. 12, 2009

(54) Individual Surgical Instruments, Surgical Instrument Set and Method for Inserting an Intraocular Lens into an Eye

(76) Inventor: Randolph L. Seil, House Springs, MO (US)

Correspondence Address: Bausch & Lomb Incorporated, One Bausch & Lomb Place, Rochester, NY, 14604-2701, US

(21) Appl. No.: 12/241,753

(22) Filed: Sep. 30, 2008

Related U.S. Application Data
(63) Continuation of application No. 10/955,213, filed on Sep. 30, 2004.

(51) Int. Cl. A61F 9/007

(52) U.S. Cl. 606/107; 128/898

(57) ABSTRACT

An instrument and method for inserting a dual optic IOL into an eye includes in one aspect a three prong forceps and in another aspect a folder for folding the IOL. The three prong forceps and folder are cooperatively configured to allow a user to first fold the IOL with the folder and then grasp and remove the folded IOL from the folder using the three prong forceps.

### CROSS REFERENCE

This application is a continuation of application Ser. No. 10/955,213 filed Sep. 30, 2004.

### BACKGROUND OF THE INVENTION

The present invention relates to surgical instruments and methods for inserting an intraocular lens (IOL) into an eye. More particularly, the present invention relates to individual surgical instruments, a surgical instrument set and method for inserting an IOL having at least one optic into an eye. The invention is particularly suited to IOLs having at least two optics.

IOLs having a single optic for placement in a person's eye have been used for many years. A variety of instruments have been proposed for inserting the IOL through an incision formed in an eye ranging from simple 2-prong forceps to injectors having a lumen through which the IOL is passed and injected into an eye in a manner similar to a syringe. Soft IOLs are usually folded or otherwise compressed to a small volume allowing the IOL to be passed through a small incision. The elastic memory of the IOL material allows the IOL to return to its original shape once placed in the eye. The most prevalent cataract removal technique today is phacoemulsification which requires an incision less than 3 mm. Phacoemulsification involves making an incision in the eye, inserting a surgical tool through the incision which breaks up the cataract lens, and finally aspirating the lens pieces back out the original incision. It is typical that the IOL is thereafter inserted through the same incision created for the phacoemulsification procedure. It is also preferred that the incision not be unnecessarily enlarged in order to insert the IOL into the eye. Accordingly, inserters have been recently designed which are capable of delivering a compressed IOL through a small (e.g., sub 3 mm) incision.

IOLs having more than one optic have been proposed in the patent literature but are not yet on the market. In a dual optic IOL, first and second optics are provided which are interconnected by one or more haptics. The two optics may alternately move toward and away from each other in response to the eye's natural accommodation movement. Accommodation is effected through the eye's ciliary muscles alternately relaxing and contracting and this movement is translated to the two IOL optics which alternately move toward and away from each other. This optic movement operates to restore accommodation to an eye. See, for example, Sarfarazi U.S. Pat. Nos. 5,275,623; 6,423,094 and 6,488,708. The insertion instruments and methods for inserting a single optic IOL are generally not suitable for inserting dual optic IOLs due to inherent design constraints (i.e., they simply were not designed for handling an IOL with two optics). There therefore exists a need for a surgical instrument and method for inserting a dual optic IOL through an incision in an eye (preferably a sub 3 mm incision).

### SUMMARY OF THE INVENTION

The present invention provides surgical instruments both individually and as a set as well as a method for inserting a dual optic IOL through an incision into an eye. It is noted that while the invention is particularly adapted for inserting a dual optic IOL into an eye, surgeons may find the instruments useful for inserting IOLs having a single optic into the eye as well and the invention should therefore not be considered as limited to use with a dual optic IOL. In a first aspect, the invention provides a three prong forcep that is designed to hold a dual optic IOL. In a second aspect, the invention provides an instrument designed for folding a dual optic IOL. As stated above, folding or otherwise compressing the IOL is required in order to pass the IOL through a small incision in the eye. In a third aspect, the invention provides an instrument set comprising the folder and the forceps which are cooperatively designed such that the forceps can receive the folded IOL from the folder. The surgeon then uses the forceps to insert the folded IOL through an incision in the eye. In a fourth aspect, the invention provides a method for inserting an IOL into an eye.

### BRIEF DESCRIPTION OF THE DRAWINGS

FIG. 1 is a plan view of an exemplary embodiment of a dual optic IOL;

FIG. 2 is a cross-sectional view thereof as taken generally along the line 2-2 in FIG. 1;

FIG. 3 is a perspective view of an embodiment of a three prong forceps;

FIG. 4 is a perspective view of an embodiment of an IOL folder;

FIG. 5 is a side elevational view thereof;

FIG. 6 is a bottom plan view thereof;

FIG. 7 is a front elevational view thereof;

FIG. 8 is a top plan view thereof;

FIG. 9 is a side elevational view of the three prong forcep of FIG. 3;

FIG. 10 is a top plan view thereof;

FIG. 11 is an enlarged, fragmented view of the distal portion of the folder showing the dual optic IOL in position and ready to be folded;

FIG. 12 is the view of FIG. 11 showing the folder in the process of folding the lens;

FIG. 13 is the view of FIG. 12 showing the dual optic lens completely folded by the folder;

FIG. 14 is a top plan view of FIG. 11;

FIG. 15 is a top plan view of FIG. 13;

FIG. 16 is a perspective view of FIG. 11;

FIG. 17 is a perspective view of FIG. 13;

FIG. 18 is an enlarged, partially fragmented, perspective view of the folder and forceps and the manner of placing the dual optic IOL onto the folder using the forceps;

FIG. 19 is the view of FIG. 18 showing the manner of grasping the folded dual optic IOL from the folder with the forceps;

FIG. 20 is the view of FIG. 19 showing the folded dual optic IOL grasped by the forceps following removal thereof from the folder;

FIG. 21 is a front elevational view of FIGS. 13 and 17 of the folder with the IOL having been folded by the folding arm which is shown in cross-section;

FIG. 22 is a front elevational view of FIG. 21 showing the folding arm cross section and in the raised position;

FIG. 23 is a front elevational view of FIG. 19 showing the prongs of the forceps in cross-section; and

FIG. 24 is the view of FIG. 23 showing the prongs being squeezed together to grasps the folded lens.

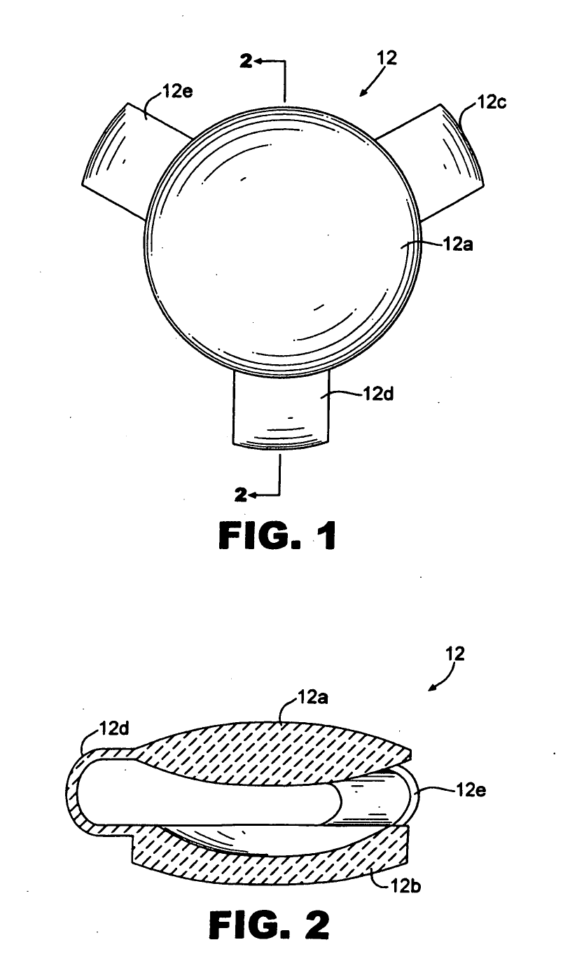

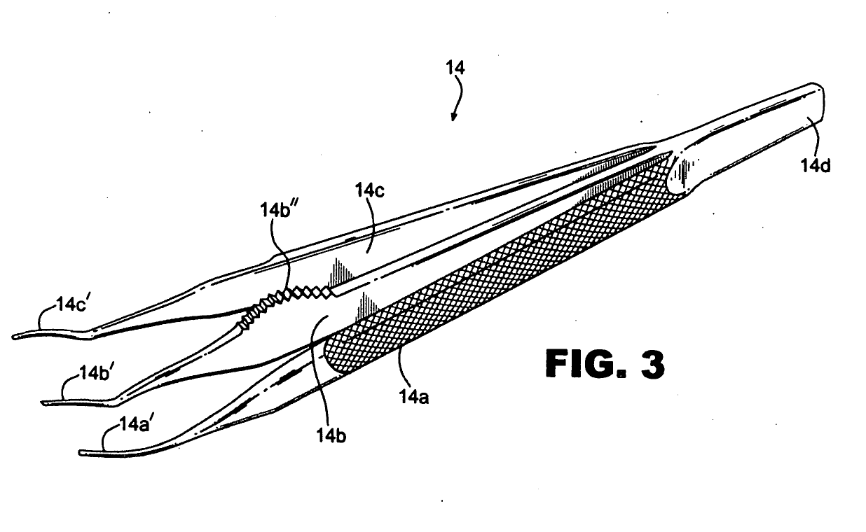

### DETAILED DESCRIPTION OF THE PREFERRED EMBODIMENT

Referring now to the drawings there is seen in the various figures a folder 10 for folding a dual optic IOL 12, and a three prong forceps 14 used to grasp the folded IOL and insert it into an eye (not shown). It is understood that the configuration of IOL 12 shown and described herein is merely for the purpose of description, it being understood that the folder and forceps may be used with other IOL designs including single and multiple optic and haptic designs. In the embodiment shown in the figures, IOL 12 has first and second optics 12a, 12b interconnected by three haptics 12c, d, and e.

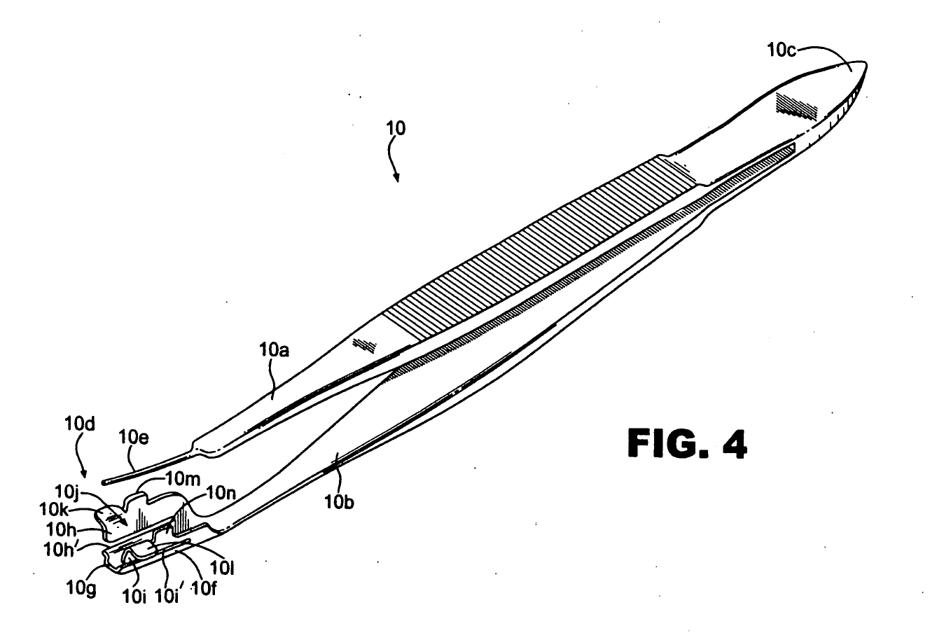

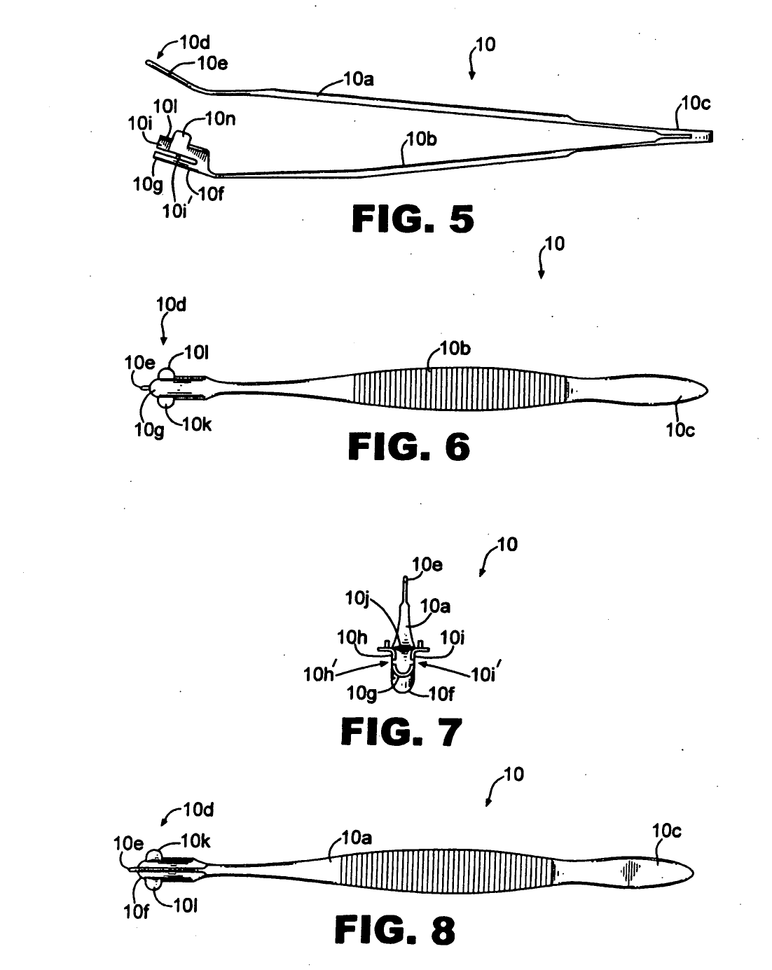

In a first aspect of the invention, a folder 10 is provided operable to fold IOL 12 in the manner to be described. Referring to FIGS. 4-8, folder 10 is seen to include first and second arms 10a, 10b connected at a proximal portion 10c, extending to a distal portion 10d with the arm terminal ends 10e, 10f in their normally spaced condition. Arms 10a, 10b are spring loaded at proximal portion 10c such that a user may squeeze terminal ends 10e, 10f together (e.g., between a finger and thumb). Upon release of the squeezing force, the terminal ends 10e, 10f return to their normally spaced condition shown in FIGS. 4-8. Arm terminal end 10e is configured as a slender folding bar while terminal end 10f is configured as a lens receiver having a bottom 10g and opposite side walls 10h, 10i defining an open top 10j facing spaced terminal end 10e. A longitudinally extending slit 10h′, 10i′ is formed in each side wall 10h, 10i, respectively, therethrough the outer prongs of forceps 14 may extend as will be described more fully below. A haptic rest 10k, 10l is provided on each side wall 10h, 10i above slits 10h′, 10i′ whereon two haptics 12c, 12d may rest and thus support the IOL 12 thereon prior to the folding operation. A side wall extension 10m, 10n may further be provided proximal to and adjacent haptic rests 10k, 10l, respectively, to provide a visual and/or physical locating means for the haptics.

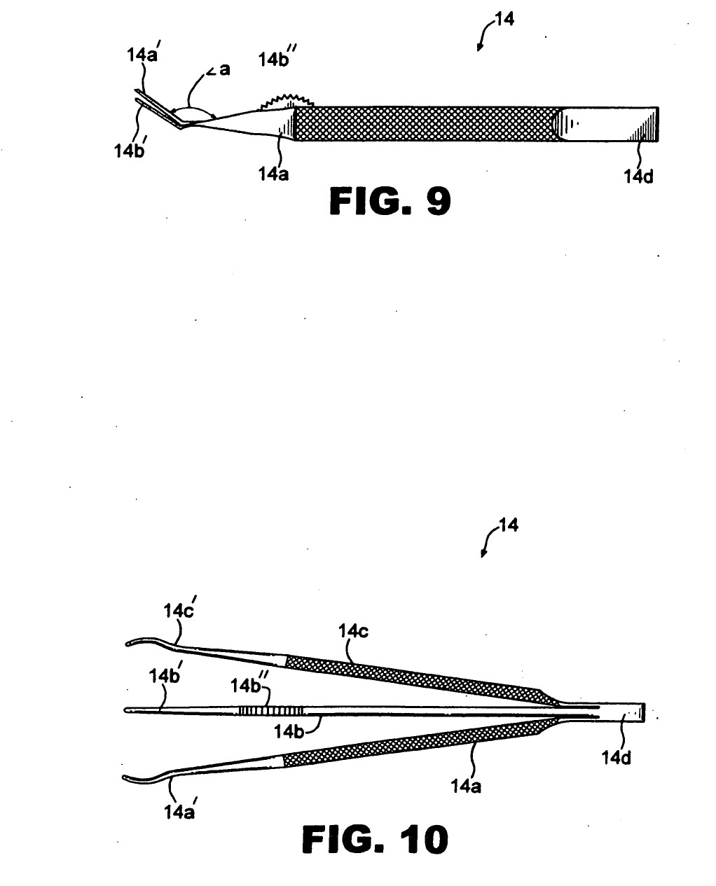

In a second aspect of the invention, a three prong forceps 14 is provided as seen best in FIGS. 9 and 10. First, second and third prong arms 14a, 14b and 14c individually extend from a proximal portion 14d and are spring biased in the normally open position seen best in FIG. 10. The distal, working prong tips 14a′, 14b′ and 14c′ extend from respective prong arms 14a, 14b and 14c and may be angled relative thereto as shown if desired. In the embodiment shown herein, prong tips 14a′, 14b′ and 14c′ extend at an obtuse angle “a” from respective prong arms 14a, 14b, 14c. Furthermore, outer prong tips 14a′, 14c′ may bow outwardly as seen best in FIG. 10 to provide a gentle holding force on the folded IOL 12. To operate forceps 14, a user presses the outer prong arms 14a and 14c toward one another where they abut center prong arm 14b. The center prong tip 14b′ itself is preferably offset from outer prong tips 14a′, 14c′ such that when the three prongs are pressed together, center prong tip 14b′ is offset from outer prong tips 14a′, 14c′ as best seen in FIG. 24. A saw-toothed finger rest 14c″ may be provided on center prong arm 14b if desired to help manually control the forceps 14. A knurled surface may also be applied to the outwardly facing surfaces of outer prong arms 14a, 14c for the same reason.

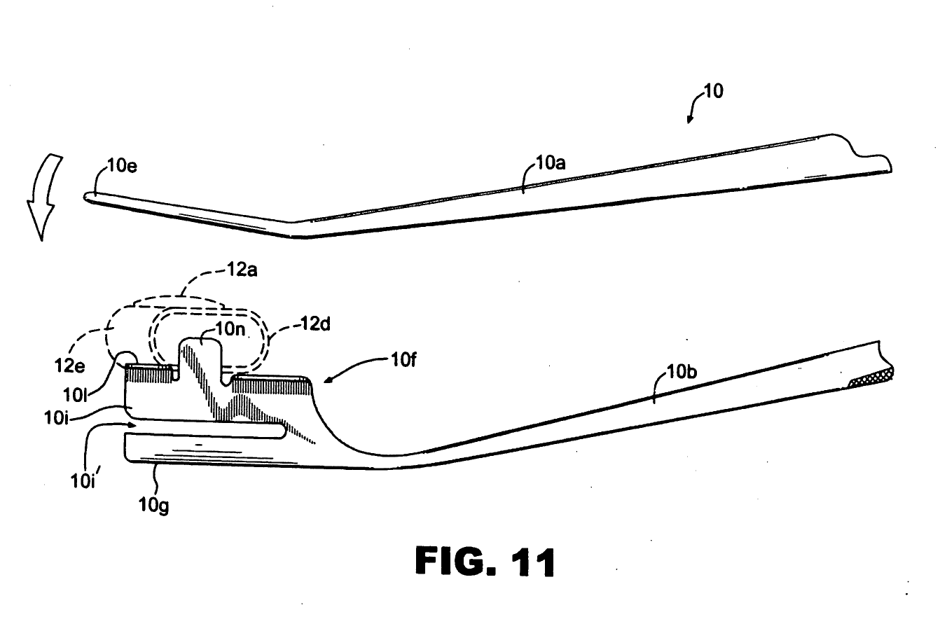

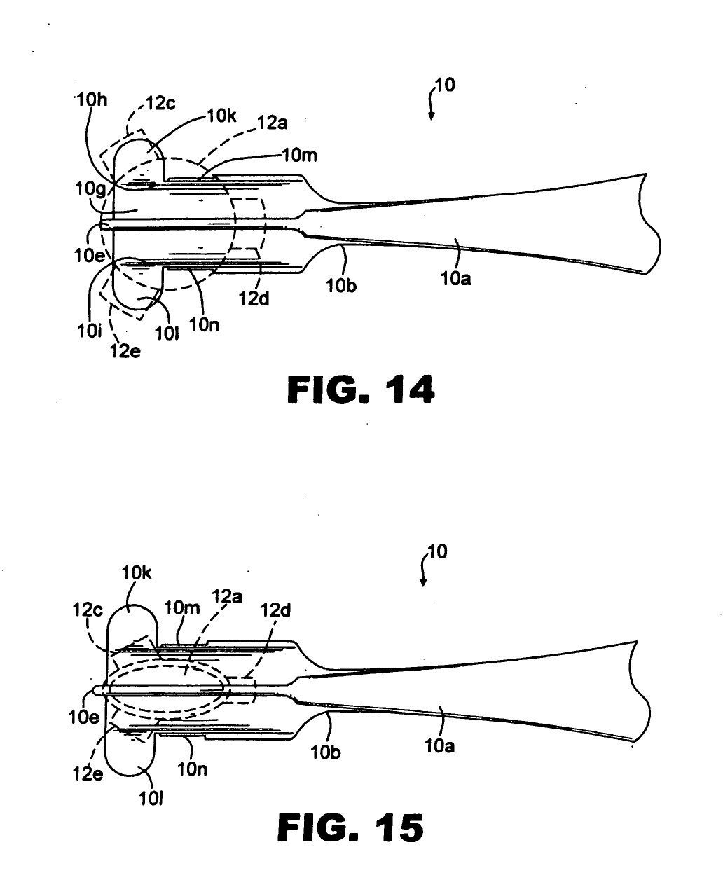

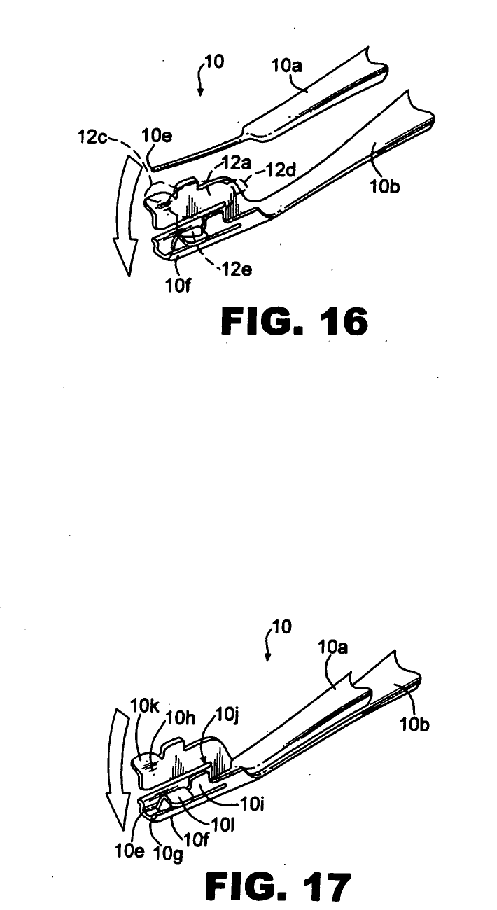

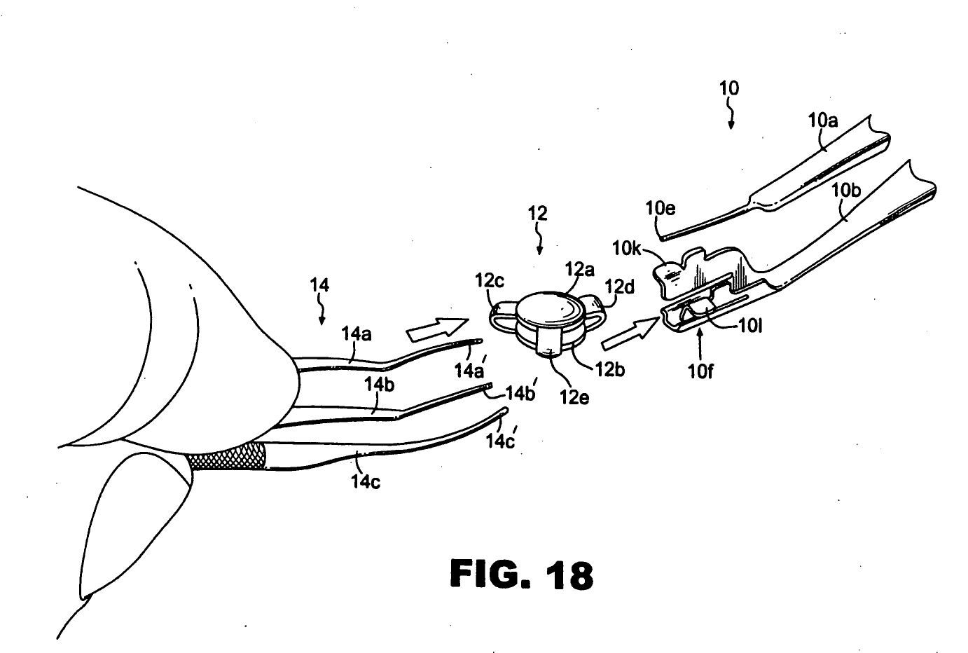

Discussion is now turned to a third aspect of the invention comprising an instrument set comprising folder 10 and forceps 14. FIGS. 11-17 and 21-22 show the folding of the IOL 12 in folder 10. FIG. 18 illustrates in spaced relation IOL 12 to be placed on folder 10 and, once folded thereby, removal with forceps 14. FIGS. 11, 14 and 16 illustrate how IOL 12 is initially positioned on folder 10. Particularly, two haptics 12c, 12e are placed upon haptic rests 10k, 10l, respectively, with third haptic 12d located proximally relative thereto generally along the axis of the folding bar 10e. Forceps 14 may be used to initially place IOL 12 on folder 10 if desired. Side wall extensions 10m, 10n provide additional IOL visual and/or physical haptic locating means although it is not required that the haptics 12c, 12e actually touch the side extensions 10m, 10n, respectively. In the initially loaded position of IOL 12 on folder 410, the lower-most optic 12b is suspended between side walls 10h, 10i and upper-most optic 12a is raised above haptic rests 10k, 10l as seen best in FIG. 11. The perimeter of the lower-most optic 12b may locate on haptic rests 10k, 10l although it is preferred this may vary according to IOL size and type relative to the spacing of the haptic rests of folder 10.

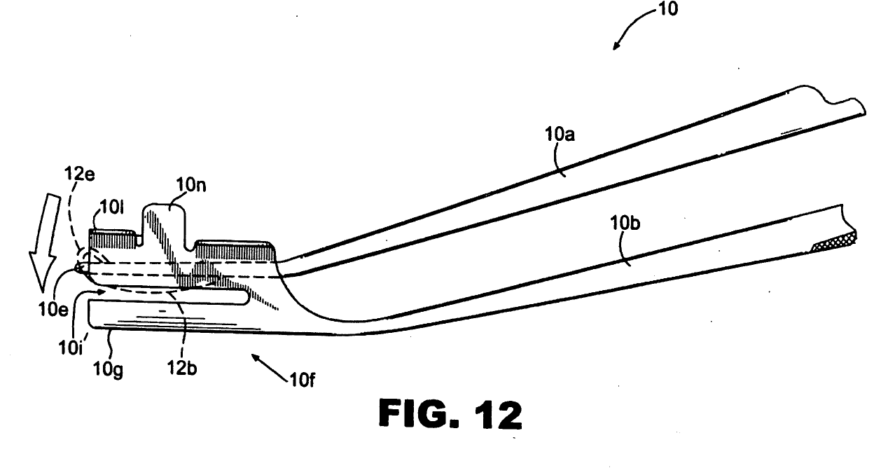

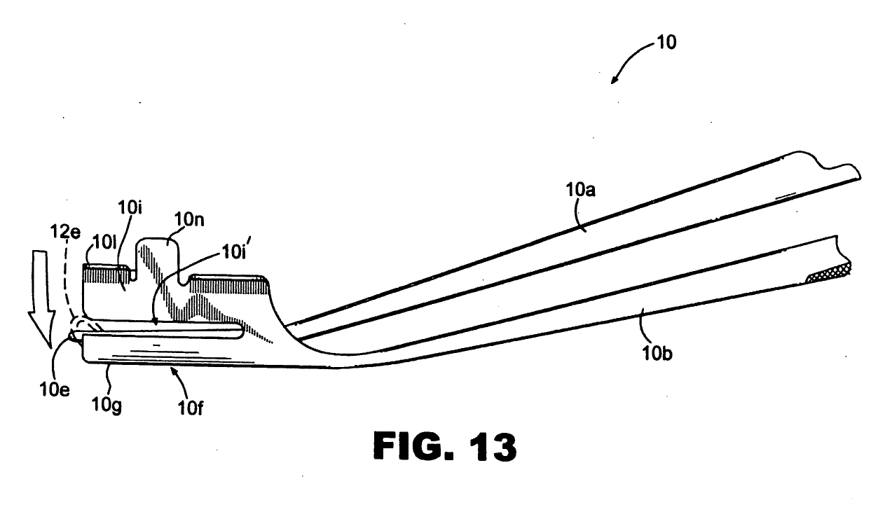

Once the IOL 12 is properly positioned on folder 410 as described above, the user presses upon folding arm 10a in the direction of arm 10b whereupon the folding bar 10e engages upper-most optic 12a substantially along the center thereof. Upon continued advancement of folding bar 012e toward bottom wall 10g, upper-most optic 12a will press against lower-most optic 12b with simultaneous outward bending of haptics 12c, 12d and 12e (see FIG. 12). The advancement of folding bar 10e is then continued until the IOL optics 12a, 12b are compressed between bottom wall 10g and folding bar 10e (see FIGS. 13 and 21). The pressure exerted on folding bar 10e should not be excessive as this may damage the IOL optics. Once the optics 12a, 12b have compressed together as seen in FIG. 21, pressure on folding bar 10e should be released whereupon it will spring back to the raised position seen in FIG. 21. The IOL 12 will remain in the folded condition as seen in FIG. 22. This concludes the IOL folding step.

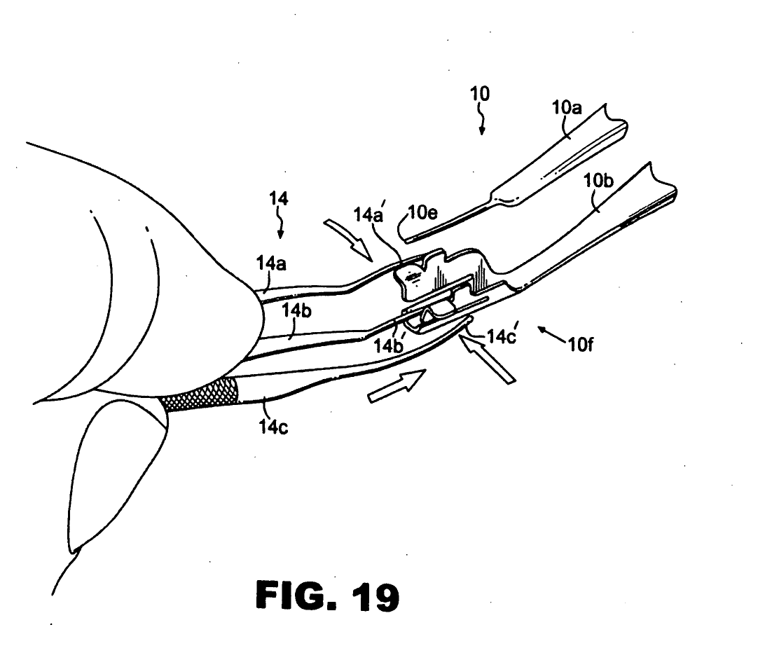

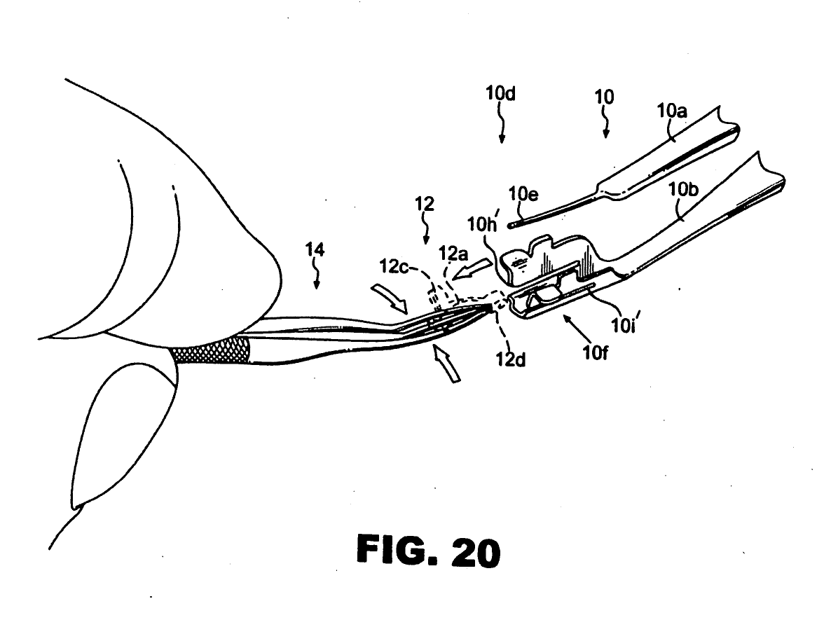

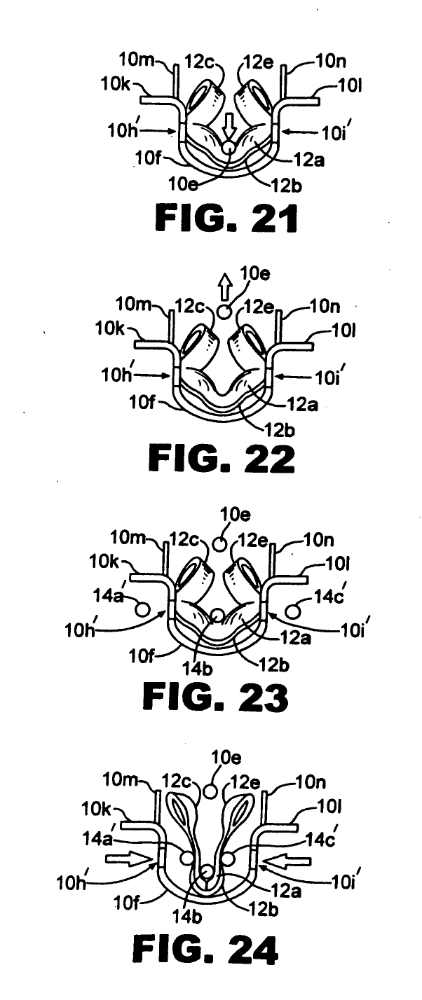

The next step is the IOL transfer step where the IOL 12 is taken from the folder 410 using the forceps 14. While holding the folder 10 with folded IOL 12 therein in one hand, the user takes forceps 14 in the other hand and removes the folded IOL 12 from the folder 10 as follows. As seen in FIGS. 19 and 23, the forceps 14 is brought to an initial position relative to the folder 10 where center prong tip 14b′ is carefully extended into the fold of IOL optics 12a, 12b and outermost prong tips 14a′, 14c′ are positioned outwardly of slits 10h′, 10i′. Care must be taken by the user not to prick the IOL 12 with the tip of the center prong 14b′ during insertion into the IOL fold. With the center prong tip 14b′ located within the IOL fold, the user begins to squeeze outermost prong arms 14a, 14c and thus also respective prong tips 14a′, 14c′ together whereupon the prong tips 14a′, 14c′ pass through slits 10h′, 10i′, respectively. Squeezing is continued until the prong tips 14a′, 14c′ engage the IOL 12 as seen in FIG. 24. As seen, the center prong tip 14b′ is offset as stated above and located slightly below outermost prong tips 14a′, 14c′. This provides a secure hold on folded IOL 12. With the IOL 12 folded and held by the forceps as seen in FIG. 24, the user raises the forceps in the direction away from bottom wall 10g through opening 10j and clears the folder 10 which may then be placed aside (see FIG. 20). It is noted the distance between side walls 10h, 10i is larger than the width of said first and third prongs 14a′, 14c′ when they are squeezed together to provide the clearance necessary to remove the forceps out of IOL receiver 10f. It is furthermore noted the folding step may be preformed by a nurse who then hands off the folder with folded IOL therein to the surgeon who then removes the folded IOL from the folder using the forceps. With the surgeon holding IOL 12 in the folded condition with forceps 14, the IOL 12 may be inserted through an incision into an eye using appropriate surgical knowledge and procedure. Once positioned in the eye, the forceps are withdrawn from the eye in the usual manner. The IOL 12 opens back to its original, unfolded condition in the eye by virtue of its own elasticity and/or through manipulation of the IOL in-situ by the surgeon using appropriate surgical instrumentation. It is noted that instead of using the forceps to directly insert the IOL into an eye, a surgeon may prefer to use the forceps to transfer the IOL into an appropriate injector (not shown) which is then used to insert the IOL directly into an eye.

There is thus described a novel IOL folder and forceps both individually as well as a set for inserting an IOL into an eye.

What is claimed is:

1. A method of folding a dual optic IOL comprising a first optic having a first center and a second optic having a second center, the centers being aligned, the method comprising:
compressing the first optic and the second optic such that, after compressing, the center of the first optic and the center of the second optic are aligned.

2. The method of claim 1 wherein, after the step of compressing, the first presses against the second optic.

3. The method of claim 1, wherein the first optic and second optic are both folded along respective centerlines.

3. The method of claim 1, wherein after the step of compressing, the first optic is folded such that peripheral portions of the first optic are displaced towards the second optic.

4. The method of claim 1, wherein the step of compressing causes the IOL to achieve a folded state, the method further comprising inserting the IOL into an eye while the first optic and the second optic are in the folded state.

5. The method of claim 1, wherein the step of compressing causes the IOL to achieve a folded state, the method further comprising holding the IOL with a forceps while the first optic and the second optic are in the folded state.
